(19) United States
(12) Patent Application Publication
(10) Pub. No.: US 2008/0097279 A1
(43) Pub. Date: Apr. 24, 2008

(54) Cosmetic Apparatus

(76) Inventor: Shigemi Sugawara, Aomori-ken (JP)

Correspondence Address: KANESAKA BERNER AND PARTNERS LLP, 1700 DIAGONAL RD, SUITE 310, ALEXANDRIA, VA, 22314-2848, US

(21) Appl. No.: 11/661,750

(22) PCT Filed: Jul. 13, 2005
(86) PCT No.: PCT/JP05/12923

§ 371 Date: Apr. 11, 2007

(30) Foreign Application Priority Data
Sep. 7, 2004 (JP) 2004-260106
May 11, 2005 (JP) 2005-138434

(51) Int. Cl. A61N 1/00; A61M 37/00; A61B 18/18

(52) U.S. Cl. 604/20; 604/25; 606/41

(57) ABSTRACT

To provide a cosmetic apparatus capable of eliminating the generation of harmful substances to a skin. An electrode part (2) is formed, by binding carbon fibers, at the tip of a high-tension wire (3) supplying a high-tension from a high-tension power supply part (17) thereto. The electrode part (2) is disposed in a soft tube (4) so that the tip (2a) thereof is positioned recessedly from the tip skin contact part (4a) of the soft tube (4). The electrode part (2) is disposed so that a clearance is provided between the high-tension wire (3) and the internal surface of the soft tube (4), and connected to an air supply means so that air can be blown out from the tip skin contact part (4a) of the soft tube (4). A hard cylindrical body (5) is fitted to and covers the tip peripheral part of the soft tube (4). A liquid-impregnated member (6) is fitted to the opened tip skin contact part (4a) of the soft tube (4), and negative ions are radiated to the skin of a face and an arm while pressing it against the skin.

### TECHNICAL FIELD

The present invention relates to a beauty apparatus for performing improvement of flecks and wrinkles of skin of a face and arms and epilation and the like.

### BACKGROUND ART

In recent years, it is empirically known that negative ions are effective for the sake of improving skin troubles, such as flecks, bruises and acnes of a face and arms. It is considered that this is because active oxygen as one of the causes of flecks, bruises and the like can be neutralized and eliminated by the negative ions, and also because ATPase of a skin cell membrane is activated by injecting the negative ions to enhance an ATP synthetic function of the cell membrane and also enhance ion permeability thereof so that extra sodium ions and calcium ions remaining in the cells move into blood, which adjusts an ion balance in the cells and activates the cells. And there are proposals of various apparatuses for irradiating the skin with the negative ions and apparatuses for beauty for the purposes of such skin-beautifying effects.

As an example of such apparatuses, there is a known apparatus, for instance, for performing iontophoresis by providing a broad treatment contact portion at an end of an end treatment tool of an iontophoretic device for the sake of introducing ions to the skin of a treatment area (Patent Document 1).

As another example, there is a skin-beautifying apparatus composed of a skin pressing portion for being pressed against a desired area of the skin and an ion wind blowing portion for blowing an ion wind including the negative ions on the desired area which is to be pressed or being pressed by the skin pressing portion, where the skin pressing portion is composed of a member including ceramics for emitting far-infrared rays, a member including germanium or a member including tourmaline (refer to Patent Document 2).

Patent Document 1: Japanese Utility Model No. 3096200

Patent Document 2: Japanese Patent Laid-Open No. 2004-148107

However, there was a problem, as to a conventional beauty apparatus which was used by directly pressing an electrode against the skin, that it caused a sharp pain on energization and inflicted suffering on a user undergoing the treatment.

There was also a problem that such a method of pressing an electrode required a fine adjustment as to how to press the electrode, its angle, voltage and the like so that its operability was bad.

To generate the negative ions, a corona discharge method is conventionally used, where the negative ions are generated by discharging electrons from a high-voltage negative electrode to a high-voltage positive electrode. In the case of this method, however, there was a problem that not only positive ions and ozone but also nitrogen oxides which are harmful to the skin are generated other than the negative ions, and they even caused skin troubles in addition to reducing beauty effects.

As for the apparatuses using the corona discharge, an ion output diode and an ion output needle (silver of purity 99.999), there was a problem that the amount of generated negative ions was too small to have sufficient beauty effects.

Furthermore, when performing the iontophoresis, there are cases where it becomes necessary to blow the ion wind on the skin while applying an essence including a moisturizer, collagen, vitamins and the like to the skin. In such cases, the conventional apparatus required the treatment to be performed with the iontophoretic device in one hand and a piece of cotton or the like including the essence in the other hand. Therefore, there were problems that spots for applying the essence became uneven, the essence could not be applied enough to have the beauty effects, working efficiency was reduced due to bad operability, and the like.

The present invention has been made to solve the problems, and an object thereof is to provide a beauty apparatus which is painless for a person undergoing the treatment and has higher beauty effects.

### DISCLOSURE OF THE INVENTION

To attain the object, the present invention provides a beauty apparatus comprising: electrodes; a power supply for applying voltage to the electrodes; a high-resistance electric conductor for electrically connecting the electrodes with the power supply; and a blower for blowing air toward the electrodes.

The electric conductor composed of a high-resistance element may also be composed of a macromolecular resistive element. The macromolecular resistive element may also be Teflon (registered trademark).

The electrodes may also be carbon fibers.

The electric conductor and electrodes may also be placed inside a nonconductive hollow tube so as to have the air from the blower blown on the electrodes by the hollow tube.

It may also have a configuration in which a gap is provided between the electrodes and an end of the tube so that a liquid-impregnated member can be held in the gap.

Furthermore, the blower may also supply the air through a gas permeation membrane which preferentially transmits oxygen. The tube may also be configured by including silicon.

It may further comprise a positive ion generating apparatus for generating positive ions and blowing the positive ions on a treatment surface of the skin and an ozone generating apparatus for generating ozone and blowing the ozone on the treatment surface of the skin.

According to the present invention, the electrodes do not directly contact the skin so that no suffering such as pain is inflicted on a person undergoing the treatment. In addition, it can enhance the beauty effects of generating more negative ions. Furthermore, it can suppress generation of the nitrogen oxides which are harmful to the skin not to mention positive ions and ozone so that it can prevent the beauty effects from being blocked.

### BRIEF DESCRIPTION OF THE DRAWINGS

FIG. 1 is a sectional view of an ion probe according to this example;

FIG. 2 is a plan view for describing a structure of a beauty apparatus according to this example;

FIG. 3 is a wiring diagram of the beauty apparatus according to this example;

FIG. 4 is a schematic diagram for describing an air flow of the beauty apparatus according to this example;

FIG. 5 is a diagram schematically showing how the discharge is performed from an electrode according to this example; and

FIG. 6 is a diagram showing a principle of a positive ion generating apparatus according to another example.

### DESCRIPTION OF SYMBOLS

S . . . power switch, V . . . vacuum button, 1 . . . negative ion irradiation apparatus, 2 . . . electrode portion, 3 . . . high voltage wiring, 4 . . . flexible tube, 4a . . . end skin contact portion, 5 . . . hard tubular element, 6 . . . liquid impregnated member, 7 . . . clearance member, 8 . . . gas permeation membrane (oxygen enrichment membrane), 9 . . . ozone probe, 10 . . . beauty apparatus, 12 . . . negative ion injection start button, 13 . . . negative ion injection time setting timer, 14 . . . negative ion injection time setting timer operation indicator lamp, 15 . . . negative ion injection air adjustment knob, 20 . . . air suction opening, 92 . . . ozone injection start button, 93 . . . ozone injection time setting timer, 94 . . . ozone injection time setting timer operation indicator lamp, 95 . . . ozone injection air adjustment knob, 96 . . . ozone amount adjustment knob.

### BEST MODE FOR CARRYING OUT THE INVENTION

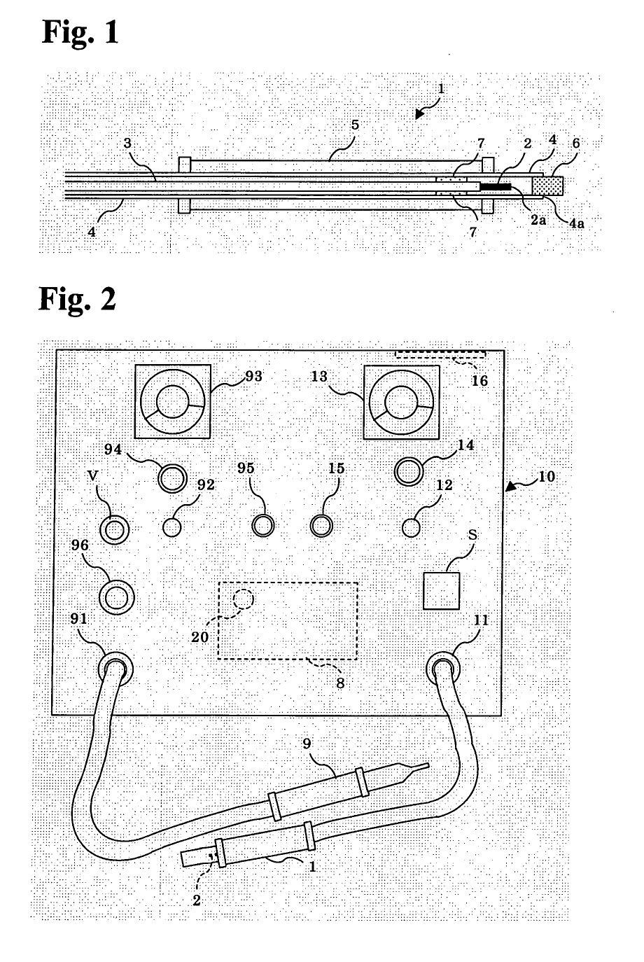

Hereunder, a beauty apparatus proper 10 according to an example of the present invention will be described with reference to FIGS. 1 to 3.

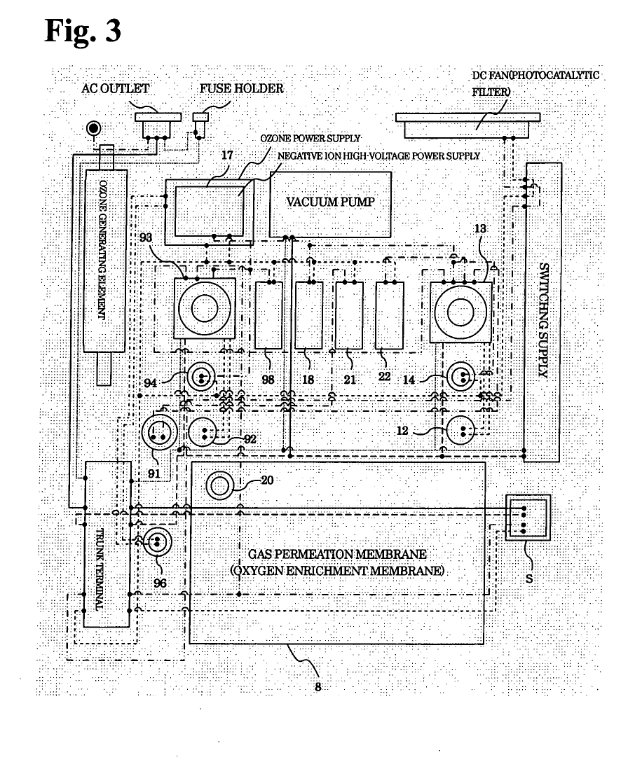

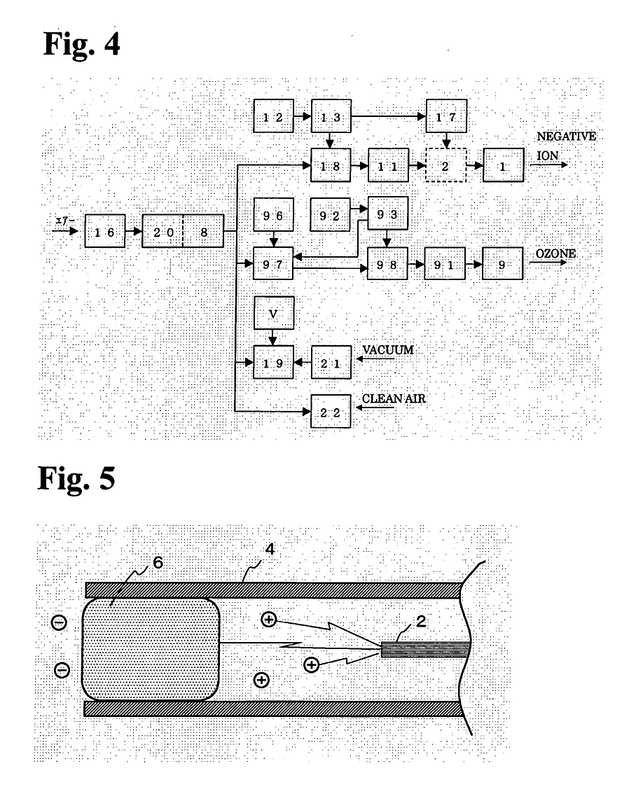

FIG. 1 is a sectional view of an ion probe according to this example. FIG. 2 is a plan view for describing a structure of the beauty apparatus according to this example. FIG. 3 is a wiring diagram of the beauty apparatus according to this example.

First, a configuration of the beauty apparatus proper will be described with reference to FIGS. 2 and 3.

In FIGS. 2 and 3, the beauty apparatus proper 10 including a probe 1 of this example comprises a power switch S, a vacuum button V, a negative ion injection start button 12, a negative ion injection time setting timer 13, a negative ion injection time setting timer operation indicator lamp 14, a negative ion injection air adjustment knob 15, a negative ion high-voltage power supply 17, a negative ion electromagnetic valve 18, an air suction opening 20, a suction electromagnetic valve 21, a spout electromagnetic valve 22, an ozone injection nozzle 91 including an ozone probe 9, an ozone injection start button 92, an ozone injection time setting timer 93, an ozone injection time setting timer operation indicator lamp 94, an ozone injection air adjustment knob 95, an ozone amount adjustment knob 96 and an ozone electromagnetic valve 98.

The power switch S is a master electrical switch of the beauty apparatus proper 10. If the power switch S is turned on, it is electrically connected to an external alternating-current source to be able to energize each of the portions.

The vacuum button 91 is a button for controlling a vacuum operation. Energization of the suction electromagnetic valve 21 is controlled by the vacuum button 91 so that a start and a stop of a suction operation can be controlled from the ozone probe 9.

The ozone probe 9 is a jet orifice for injecting the ozone, and has an acid-resistant and chemical resistant capsule in an end-pointed shape mounted at its end. As it is in such an end-pointed shape, it is possible to pointedly inject the ozone at flecked portions and the like of the skin.

A flexible tube 4 on the negative ion side has the negative ion probe 1 mounted at its end, and is a tube for blowing the air into the probe 1. The tube 4 may be composed of silicon or the like. As the end of the flexible tube 4 is projected further to the end than an electrode portion 2, a gap is provided between the electrode portion 2 and the end of the flexible tube 4. An impregnated member which has permeability and is nonconductive such as cotton is held in the gap so as to discharge from the electrode with the cotton impregnated with a solution used for beauty.

The operation button 12 is a button for performing operations of injecting and stopping the negative ions from the probe 1 by controlling the energization to the negative ion high-voltage power supply 17 and the negative ion electromagnetic valve 18.

The negative ion injection time setting timer 13 is a timer for setting the time period for injecting the negative ions. The negative ion injection time setting timer 13 performs the energization to the negative ion high-voltage power supply 17 and the negative ion electromagnetic valve 18 just for a set time period, and cuts off the energization thereto when the set time comes to stop injecting the negative ions.

The negative ion injection time setting timer operation indicator lamp 14 is composed of an LED. If the operation button 12 is turned on to be in an injection state, the negative ion injection time setting timer operation indicator lamp 14 is energized and lights up in green. If the operation button 12 is turned off or if it becomes the set time of the timer, the energization is cut off and it blacks out.

The negative ion adjustment knob 15 is a knob for adjusting the amount of the negative ions to be injected. The negative ion adjustment knob 15 constitutes a slidax, and it can adjust the amount of the negative ions to be injected by adjusting the voltage supplied to the negative ion high-voltage power supply 17 from the alternating-current source.

The negative ion high-voltage power supply 17 supplies high voltage to the electrode portion 2. In this example, the negative ion high-voltage power supply 17 can supply the voltage of 6.2 KV at the maximum.

The negative ion electromagnetic valve 18 is an electromagnetic valve of which energization is controlled by an operation of the negative ion injection start button 12 to open and close the valve so as to control supply of the air supplied from a pump to the flexible tube 4.

The air suction opening 20 is a suction opening of injected air. The air suction opening 20 has a gas permeation membrane 8 mounted thereon. As for the gas permeation membrane 8, it is possible to use an oxygen enrichment membrane (PanaO₂) manufactured by Matsushita Electric Industrial Co., Ltd. For instance, which can reduce the nitrogen in the air by approximately 10% in addition to separating rubbish and dust sucked in. It is thereby possible to prevent nitrogen oxides from being generated on generating the negative ions.

The suction electromagnetic valve 21 is configured to open and close a suction valve by an operation of the vacuum button 91, whereby a suction operation is controlled by the pump.

The spout electromagnetic valve 22 is a valve for controlling the air injected from the negative ion probe 1.

The ozone probe 9 is a jet orifice for injecting the ozone. The ozone probe 9 is composed of a flexible material such as silicon, and the jet orifice thereof can be freely manipulated.

The ozone injection start button 92 is a button for starting ozone injection by controlling the energization to an ozone power supply and the ozone electromagnetic valve 98.

The ozone injection time setting timer 93 is a timer for setting the time period in which the ozone is injected, which can stop injection of the ozone by cutting off the energization to the ozone power supply when the set time comes.

The ozone injection time setting timer operation indicator lamp 94 is composed of an LED. If the ozone injection time setting timer 93 is turned on, the ozone injection time setting timer operation indicator lamp 94 is energized and lights up in green. And it has the energization cut off and blacks out when the set time comes.

The ozone injection air adjustment knob 95 is intended to adjust the amount of the air on injecting the ozone. The ozone injection air adjustment knob 95 is composed of a slidax. As it controls the voltage supplied to the electromagnetic valve 98, if the knob is turned clockwise, it loses momentum and the amount of injected ozone is reduced. If the knob is fully turned, the ozone injection stops.

The ozone amount adjustment knob 96 is intended to adjust the generated amount (concentration) of the ozone. The ozone amount adjustment knob 96 constitutes a slidax, and is capable of adjusting the ozone concentration by adjusting the voltage supplied to the ozone power supply. If the ozone amount adjustment knob 96 is turned clockwise in this example, the ozone concentration becomes higher so that the injection can be performed up to the ozone concentration of 10 ppm at the maximum.

The ozone electromagnetic valve 98 is an electromagnetic valve for opening the valve and supplying the ozone for the time period set by the ozone injection time setting timer 93 by the operation of the ozone injection start button 92.

Next, details of the probe 1 for injecting the negative ions will be described with reference to FIG. 1.

As shown in FIG. 1, the probe 1 of this example comprises the electrode portion 2 for generating the negative ions by having a high voltage applied thereto, a high voltage wiring 3 for electrically connecting the electrode portion 2 with the negative ion high-voltage power supply 17 (shown in FIG. 3) and the hollow flexible tube 4 made of silicon having the electrode portion 2 and high voltage wiring 3 placed therein. A cylindrical portion around the end of the flexible tube 4 is inserted into a hard tubular element 5 composed of an aluminum pipe and is thereby protected, where the hard tubular element 5 constitutes a handle of a person who performs the operation.

The high voltage wiring 3 is connected to the electrode portion 2. The electrode portion 2 is constituted by bundling multiple carbon fibers as a load resistance element.

The electrode portion 2 has its end 2a constituted further back from a skin contact portion 4a at the end of the flexible tube 4, and has a gap formed between the end of the electrode portion 2 and the skin contact portion 4a at the end of the flexible tube 4. And it can hold a liquid impregnated member 6 such as cotton impregnated with an essence such as collagen in the gap portion.

Furthermore, the electrode portion 2 has a clearance member 7 made of cotton and having permeability between the high voltage wiring 3 and the inside of the flexible tube 4, and has a clearance provided between the flexible tube 4 and a support of the electrode portion 2. Thus, the air blown in can pass through the clearance member 7 to be blown on the electrode portion 2.

The electrode portion 2 may also be connected to the high voltage wiring 3 via a support of Delrin (registered trademark), Teflon (registered trademark) or the like. In this case, the clearance member may also be placed between the support and the inside of the flexible tube 4.

The high voltage wiring 3 is composed of a high-resistance electric conductor. The high voltage wiring 3 may be composed of Teflon (registered trademark) as a polymeric conductor for instance. Thus, according to the configuration, the high voltage wiring 3 has a higher resistivity than the electrode portion 2, and a high voltage is thereby applied to the electrode portion 2 so as to be discharged.

A base portion of the flexible tube 4 is communicated with the pump (not shown) to blast the air from the skin contact portion 4a at the end. The air is supplied through a gas permeation membrane which preferentially transmits oxygen, such as the oxygen enrichment membrane (PanaO₂) manufactured by Matsushita Electric Industrial Co., Ltd.

Next, a description will be given as to an operation procedure in the case of irradiating the skin with the negative ions by using the beauty apparatus according to this example.

1) First, it is powered on by pushing the power switch S. Thus, the pump provided inside operates.

2) Next, the negative ion injection time setting timer 13 is dialed to set the negative ion injection time.

3) The end 4a of the tube 4 is applied to the face, and the negative ion injection start button 12 is pushed. Thus, a negative ion irradiation function is activated so that the electrode portion 2 is energized and operates just for the set period of time.

4) In this case, the spout electromagnetic valve 22 is opened and closed by operating the negative ion injection air adjustment knob 15 to allow adjustment of the amount of air injected from the end 4a of the tube 4 of the probe 1.

5) Furthermore, a suction function is activated by pushing the vacuum button V. The suction function is usable only in the cases where each of the functions such as the negative ion irradiation or the ozone injection is stopping. A vacuum apparatus is also used as the tube of an ozone irradiation apparatus 9.

6) In the case of injecting the ozone, the time for using the function is set by dialing the ozone injection time setting timer 93. The end of the tube of the ozone irradiation apparatus 9 is applied to the skin, and the ozone injection start button 92 is pushed. Thus, the function of injecting the ozone is activated and stops when the set time comes.

Next, a description will be given as to the principle of discharging the negative ions from the electrode portion 2.

If a high voltage is applied to the high voltage wiring 3 from the negative ion high-voltage power supply 17, the high voltage is applied to the electrode portion 2 without being discharged from the high voltage wiring 3 since the electrode portion 2 has a lower resistivity than the high voltage wiring 3. As there is no counter electrode against the electrode portion 2 in this state, a flow of electrons is limited until the voltage applied to the electrode portion 2 exceeds a certain limiting point. Once it exceeds the limiting point, however, negative electrons are discharged as if pushed out of the electrode portion 2.

As the atmosphere constantly includes about 30% of moisture, hydrogen ions (positive ions) in the moisture are constantly suspended in the atmosphere. In addition, the positive ions exist in the atmosphere. For that reason, it is possible, without especially seeking a positive electrode, to discharge electricity by regarding the atmosphere as a virtual positive electrode. As an impedance of the electrode portion 2 is high in comparison with the impedance between the virtual positive electrode and the electrode portion 2, it becomes possible to discharge negative ion electrons.

FIG. 5 schematically shows how the discharge is performed. As shown in FIG. 5, if the air is supplied from the pump, the air includes positively charged water molecules which are suspended in the atmosphere. If the air is supplied as far as the electrode portion 2, electricity is discharged to the positively charged water molecules from the end of the electrode portion 2 so that the water molecules become negatively charged. As the impregnated member 6 includes the essence, electricity is also discharged to the impregnated member 6 from the electrode portion 2 so that the essence included in the impregnated member 6 becomes negatively charged.

The negatively charged water and essence are transmitted through the impregnated member 6 and discharged toward the skin from the end of the flexible tube 4 by the air supplied from the pump.

To discharge the negative ions, it is necessary to match a power supply voltage with the electrode portion 2. As an example, the negative ions are discharged if the voltage of the high-voltage power supply is 5 KV and a load resistance of the electrode portion 2 is 20 Ω.

And as an example, the amount of the negative ions discharged from the end of the electrode portion 2 was measured. As a result, 2 million pieces/cc or more of the negative ions were measured from the electrode portion 2 of the probe 1 of this example.

The measurement apparatus used was an ion system measurement device (MODEL KST-900) manufactured by Kobe Denpa Co., Ltd., and measurement conditions were as follows.

Measured ions: positive and negative ions, mobility 0.4 cm²/V-sec or higher

Space-charge density: difference between the number of positive ions and the number of negative ions of the total number of ions

Measurement environment: high-density environment due to ion generation in the atmosphere

Measurement range: 5 to 999900 (pieces/cc)

Sampling flow: 60 l/min

Thus, the high voltage wiring 3 was composed of a material of higher resistance than the electrode portion 2, and a high voltage was applied thereto so that the electrons were pushed out of the electrode portion 2 to allow more negative ions to be generated. Consequently, the negative ions generated in large quantity can neutralize or eliminate active oxygen and poisonous positively charged substances on the skin surface, and so a stimulus to the skin decreases to suppress inflammation of the skin. The cells of the skin can be activated, which also promotes absorption of the essence and the like and enhances the beauty effects. The essence of the impregnated member 6 is sprayed on the skin in an ionized state to promote absorption of the essence into the skin.

It is also possible, by blowing the air toward the electrode portion 2, to continuously discharge the ionized air from the end of the tube 4. A gap was provided between the electrode portion 2 and the end 4a of the flexible tube 4 to be able to hold in the gap the impregnated member 6 such as cotton capable of being impregnated with a liquid such as an essence. Therefore, it is possible to irradiate the skin with ionic air while applying the essence or the like thereto at pinpoints and efficiently. As the impregnated member 6 is a nonconductive substance, it is not directly discharged on the skin so that the beauty effects can be enhanced without causing a pain or a sense of discomfort to a user undergoing the treatment.

Furthermore, according to the above-mentioned discharge method, electricity is discharged to the positively charged water molecules. Therefore, it is possible to suppress generation of the positive ions and ozone due to corona discharge. Consequently, the ozone and nitrogen oxides are no longer unreadily emitted so that the beauty effects due to the negative ions are no longer blocked and no side-effect is generated.

The air is supplied to the suction opening of the pump through the gas permeation membrane which preferentially transmits oxygen. Therefore, it is possible to reduce nitrogen content of the air, prevent generation of the nitrogen oxides and supply clean air of which dust, dirt, bacteria and viruses are cut.

As the tube 4 is composed of silicon, the tube 4 can be flexible enough to have an ion air irradiation portion freely replaced, and feels pleasant enough to give no sense of discomfort to the person undergoing the treatment.

Furthermore, according to the present invention, the hard tubular element 5 is mounted on the cylindrical portion around the end of the flexible tube 4 so that the flexible tube can be easily handled without getting broken in the middle.

The present invention is not limited to one of the above-mentioned negative ion irradiation apparatuses but may also be a beauty apparatus combining the negative ion irradiation apparatus with an ozone generating apparatus for generating ozone or a beauty apparatus combining the negative ion irradiation apparatus with a positive ion irradiation apparatus for generating positive ions.

It is thereby possible to exert skin-beautifying effects not only using the negative ions but also using the negative ions and ozone or the negative ions and positive ions together.

This example described the negative ion irradiation apparatus and the beauty apparatus comprising the negative ion irradiation apparatus and an ozone injection apparatus. However, the present invention is not limited thereto but may also be the beauty apparatus comprising the negative ion irradiation apparatus and a positive ion apparatus.

Next, a description will be given as to an example of applying the beauty apparatus using the negative ions, ozone and positive ions together to an epilation apparatus. In this example, it is an apparatus wherein a positive ion generating apparatus is added to the apparatus of the above-mentioned example.

The same configuration as the above-mentioned example will be given the same symbols, and a description thereof will be omitted.

As in National Publication of International Patent Application No. 10-506554 for instance, a conventional epilation method exposes follicles by mechanically or chemically removing hair, and destroys the follicles by applying a photosensitization agent and passing a current for instance. This conventional method introduces a follicle inactive compound to the follicles by using an iontophoretic method. As for this, it is described that a current is passed to vacant follicles after removing the hair so as to destroy the follicles and perform the epilation.

However, the above-mentioned method mechanically removed the hair or chemically removed the hair by applying wax, and so it was accompanied by a pain on doing so. In the case of passing the current to the follicles thereafter, there was a problem that it was accompanied by a pain again on passing the current.

The apparatus according to this example has solved such a problem.

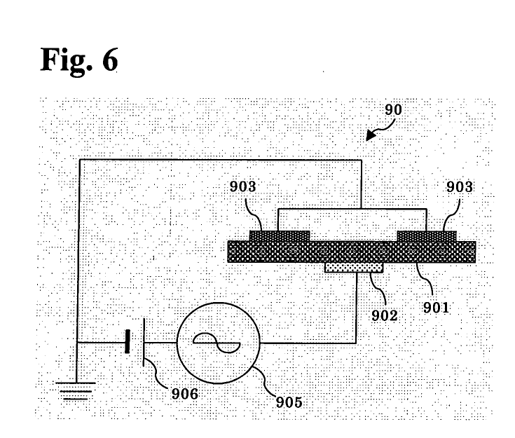

This example is configured to be able to inject the positive ions generated by the electromagnetic valve from the probe 1 and the like by providing a positive ion generating element shown in FIG. 6 and a high-voltage power supply for the positive ion generating element inside a beauty apparatus 10 of the above-mentioned example.

As for the positive ion generating element, it is an apparatus for ionizing surrounding air by placing a discharging electrode 902 on one surface of a dielectric 901 composed of a ceramic, mica or the like, placing an induction electrode 903 on the other surface, and applying an alternating-current voltage 906 and a direct-current bias voltage 905 between the discharging electrode 902 and the induction electrode 903 so as to discharge electricity (plasma).

Thus, it is possible, by applying a positive direct-current bias voltage to the discharging electrode 902, to have the negative ions absorbed by the discharging electrode 902 and take out only the positive ions which can be injected from the probe 1 or a probe 2 by operating the electromagnetic valve.

Next, an epilation method using the above-mentioned beauty apparatus will be described.

First, an epilation solution is applied to the portion of the skin to be epilated (such as an armpit, a shin or an arm). Here, the epilation solution is a solution including isoflavone, papain, a moisturizer and the like.

When the epilation solution is applied, the papain which is a proteolytic enzyme included in the epilation solution resolves hair cells and hair root cells. The isoflavone of the epilation solution acts like estrogen which is female hormone and thereby acts on hair mother cells to suppress hair growth.

Next, the ozone probe 9 is brought to the skin to be epilated, and the ozone is injected. Thus, stains and bacteria on the skin surface are removed, and the hair cells to be epilated are inactivated.

Next, the negative ion probe 1 is brought to the skin to be epilated, and the negative ions are injected. The injection of negative ions activates the skin to be epilated so that pores of that portion open to promote absorption of the epilation solution and the like. To be more specific, ATPase of a cell membrane is activated by injecting the negative ions to enhance an ATP synthetic function of the cell membrane and also enhance ion permeability thereof so that extra sodium ions and calcium ions remaining in the cells move into blood, which adjusts an ion balance in the cells and activates the cells so as to promote absorption of the epilation solution and the like.

In this case, an impregnated member such as cotton impregnated with the epilation solution can be mounted in the gap at the end of the tube.

Lastly, the electromagnetic valve is switched, and the positive ions are injected from the negative ion probe 1 to inject and implant them into the skin to be epilated. Thus, the positive ions are implanted into the hair root mother cysts and follicle cells to inactivate the hair root cells.

The hair root cells and follicle cells are destroyed by the inactivation of the hair root cells due to the implantation of the positive ions, and so the hair easily comes out. The hair growth is suppressed by inactivation action of the epilation solution and the positive ions on the hair mother cells, follicle cells and the like.

The above-mentioned example described the example of separately injecting the negative ions and the positive ions. However, they may also be simultaneously injected from the same probe 1.

1. A beauty apparatus for performing a beauty treatment to skin, comprising:
electrodes;
a power supply for applying voltage to the electrodes;
an electric conductor having a higher resistivity than the electrodes and electrically connecting the electrodes with the power supply;
a blower for blowing air toward the electrodes; and
a nonconductive hollow tube for housing the electric conductor and electrodes and passing the air from the blower, characterized in that:
a gap is provided between the electrodes and an end of the tube so that an impregnated member which has permeability and is nonconductive is held in the gap so as to energize the electrode and discharge electricity therefrom with the impregnated member impregnated with a solution used for beauty.

2. The beauty apparatus according to claim 1, characterized in that the macromolecular resistive element is Teflon (registered trademark).

3. The beauty apparatus according to claim 1, characterized in that the electrodes are carbon fibers.

4. The beauty apparatus according to claim 1, characterized in that the tube is configured by including silicon.

5. The beauty apparatus according to claim 1, characterized in that the blower supplies the air through a gas permeation membrane which preferentially transmits oxygen.

6. The beauty apparatus according to claim 1, further comprising:
a positive ion generating apparatus for generating positive ions and blowing the positive ions on a treatment surface of the skin; and
an ozone generating apparatus for generating ozone and blowing the ozone on the treatment surface of the skin.
